(19) United States
(12) Patent Application Publication
(10) Pub. No.: US 2021/0205488 A1
(43) Pub. Date: Jul. 8, 2021

(54) UV-C EMITTING FABRIC

(71) Applicant: Tru-UV, LLC, Chestertown, MD (US)

(72) Inventors: Patrick J. Shanahan, Chestertown, MD (US); James Shifrin, Chestertown, MD (US); Cyrus Etemad-Moghadam, Fallston, MD (US); Samantha Verzi, Forest Hill, MD (US)

(21) Appl. No.: 17/142,274

(22) Filed: Jan. 6, 2021

Related U.S. Application Data
(60) Provisional application No. 62/957,868, filed on Jan. 7, 2020.

(51) Int. Cl. A61L 2/26; F21V 8/00; G02B 6/10; A61L 2/10

(52) U.S. Cl. CPC A61L 2/26; G02B 6/001; A61L 2202/11; G02B 6/102; A61L 2/10; G02B 6/0006

(57) ABSTRACT

A fabric comprises a flexible fiber optic array extending within a body of the fabric and a light source affixed to the fabric and arranged to direct electromagnetic radiation into the flexible fiber optic array. The flexible fiber optic array includes at least one fiber optic cable that transmits the electromagnetic radiation emitted from the light source and that emits the electromagnetic radiation from a length of a longitudinally-extending side of the at least one fiber optic cable.

### CROSS REFERENCE TO RELATED APPLICATIONS

This application claims the benefit under 35 USC § 119(e) of U.S. Provisional Patent Application No. 62/957,868 filed Jan. 7, 2020.

### BACKGROUND

The present invention is directed to sanitizing various objects, surfaces, and the like with the use of ultraviolet light, such as ultraviolet C (UV-C) light.

It is conventional practice to sanitize objects, such as, medical instruments, stethoscopes, reflex hammers, and the like, and surfaces, or the like with the use of alcohol wipes or other wipes combined with sanitizing sprays or fluids. In addition, high heat methodologies or chemical gas atmospheres may be used for purposes of sanitizing objects, surfaces, or the like. Still further, UV-C light may be utilized for this purpose. For instance, UV light of a frequency of about 264 nm can disrupt DNA of bacteria and limit the ability of bacteria to reproduce. As such, bacteria can be eliminated to a relatively high degree (99%) from whatever object or surface that is exposed to the UV light.

### SUMMARY

Embodiments disclosed herein are directed to a fabric comprising a flexible fiber optic array extending within a body of the fabric and a light source affixed to the fabric and arranged to direct electromagnetic radiation into the flexible fiber optic array. The flexible fiber optic array includes at least one fiber optic cable that transmits the electromagnetic radiation emitted from the light source and that emits the electromagnetic radiation from a length of a longitudinally-extending side of the at least one fiber optic cable.

### BRIEF DESCRIPTION OF THE DRAWINGS

FIG. 1 is a plan view of a UV-C emitting fabric according to a first embodiment.

FIG. 2 is a plan view of a UV-C emitting fabric according to a second embodiment.

FIG. 3 is a plan view of a UV-C emitting fabric according to a third embodiment.

FIG. 4 is a side elevational view of the UV-C emitting fabric of FIG. 3.

FIG. 5 is a plan view of a UV-C emitting fabric according to a fourth embodiment.

FIG. 6 is a plan view of the UV-C emitting fabric of FIG. 5.

### DETAILED DESCRIPTION

According to an embodiment, a fabric including a flexible fiber optic array is provided that can be used to emit UV-C light or other electromagnetic radiation onto an object or surface of an object for sanitizing the object or surface. The UV-emitting fabric can be used in various forms, for instance, it can be used as a cloth to line a box, a pouch or bag, a pocket of a shirt or smock or lab coat, or it can be used in the form of a blanket or cover for being placed over an object such as a computer keyboard, microscope, lab work surface, or the like. The fabric may also be used in car headliners or ambulances/first responder vehicles or compartments used for storage of medical devices in such vehicles. Thus, in use, the fabric is utilized to emit UV-C light or other electromagnetic radiation onto an object or surface adjacent, covered, or surrounded by the fabric for purposes of sanitizing the object or surface between uses.

According to an embodiment, the fabric includes an array of fiber optic cables, threads, or the like that emits UV-C wavelength light along their length. The cables may be woven into the fabric with or without other material or the like and may be configured to emit UV-C light along their length and not just at the free end of the cable. In this manner, the flexible fiber optic cables or threads can be woven into a fabric with other material to create a blanket, cloth, or like product and can be used in various forms to create different sanitizing products.

UV-C emitting light-emitting diodes (LEDs) or like electronic devices may be used as light sources to emit and deliver UV-C light into the cables. The cables, which may be woven into a lattice form along with other material fibers (such as cotton) to form a fabric, receive the light, transmit the light along a length thereof, and project the light from the length thereof. The outer layers of the fiber optic cables may be treated or formed such that at least some of the light that is being transmitted along a part of a length of the cable may be able to be emitted from the side of the cable along its length, or at least a pre-determined portion of the length, and is not merely emitted from a free end of the cable. In this manner, the fabric is able to emit UV-C light radially from the sides of the cables in an even and substantially uniform fashion across the entire face of the fabric or a pre-determined part of the face of the fabric.

According to an embodiment, a fiber optic header or headers may be used to feed UV-C light from the LED to multiple cables projecting from the header.

According to an alternate embodiment, the UV-C light may be emitted into the cables at a location along a length of the cables (as opposed to into an end of the cables) and transmitted to another length of the cables where it is emitted from other downstream areas of the cables. One or more UV-C LEDs may be located at any of various locations in the fabric weave to maximize the amount of UV-C emitted into the cables and projected from the cables.

The flexible fabric having a flexible fiber optic array as discussed above could be used for the purpose of emitting UV-C light onto one or more objects that is/are next to, adjacent, or surrounded by the fabric. By way of example, and not by way of limitation, a box could be lined with the fabric for use in sanitizing medical instruments placed within the box, a pocket of a smock of a doctor or nurse could be formed or lined with the fabric for purposes of sanitizing any object placed into or carried within the pocket, and curtains, blankets, covers, or the like may be made of the fabric for purposes of sanitizing other surfaces and objects such as hospital rooms, furniture, surfaces, apparatus, and the like.

As also stated above, the fabric may include one or more LED UV-C light sources arranged to introduce UV-C wavelength light into the flexible fiber optic cables extending in and forming the fabric. The UV light may be introduced into the ends of the fiber optics, into the sides of the fiber optic cables, or into a header or the like. For instance, a header, such as provided in the form of a glass (or other material) plate or the like, may be used to introduce the light into the fibers and may be relatively large as compared to the many fibers to which it provides a source of light.

The fiber optic cables may be selectively treated, formed, or the like so that the cables are able to emit UV-C light along at least portions of their lengths as opposed to merely containing and transmitting the light such that it is only emitted from an opposite end of the cable. By enabling the cables to emit light in a radial direction along their length, a more usable and efficient form of fabric may be provided. Also, by not having light emitted only from ends of cables, fewer cables may be used to produce the fabric and it is not required to form sharp bends and/or to precisely aim the ends of the cables from which light is emitted.

The cables of the flexible fiber optic array may be woven to form a fabric along with other material fibers to form a UV-C emitting fabric. Thus, fibers treated so that they emit light along their length are woven into the body of the fabric so that they are appropriately located to sanitize objects adjacent or surrounded by the fabric.

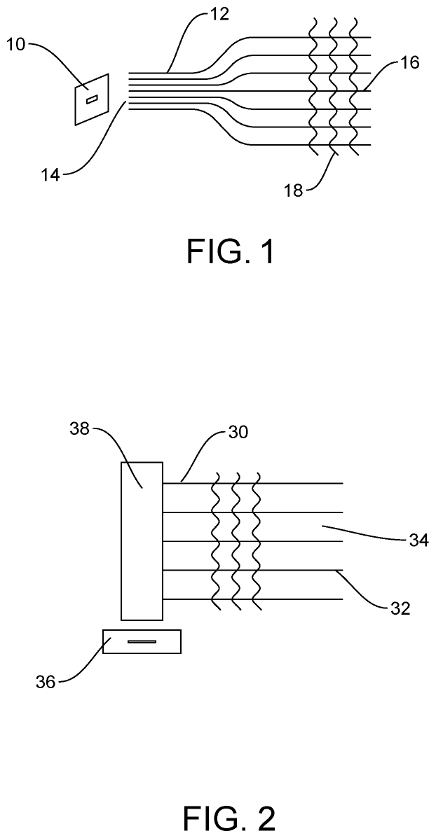

FIG. 1 shows a UV light source 10 coupled to an end 14 of a bundle of fiber optic cables 12. A section 16 of the fibers 12 remote from the light source 10 are spread out into a fabric weave 18 and are woven with other materials to form the fabric. The fiber optic cables 12 are treated or formed so that they emit UV light along at least a section of their lengths.

By way of example, the cables 12 may be provided such that they only emit UV light at selective locations along one or both faces of the fabric 18. For instance, the cables 12 may not emit light along their entire length, may emit light on only one side of the cable or face of the fabric, or may emit light radially along the cables 12 in all directions. In addition, the cables 12 may be provided such that they emit UV from the front side of the cloth or fabric, but not the opposite side.

According to another embodiment shown in FIG. 2, the ends 30 of multiple fiber optic cables 32 that form part of a fabric 34 are arranged to receive UV light from a light source 36 via a header 38 which may be a relatively larger fiber optic cable.

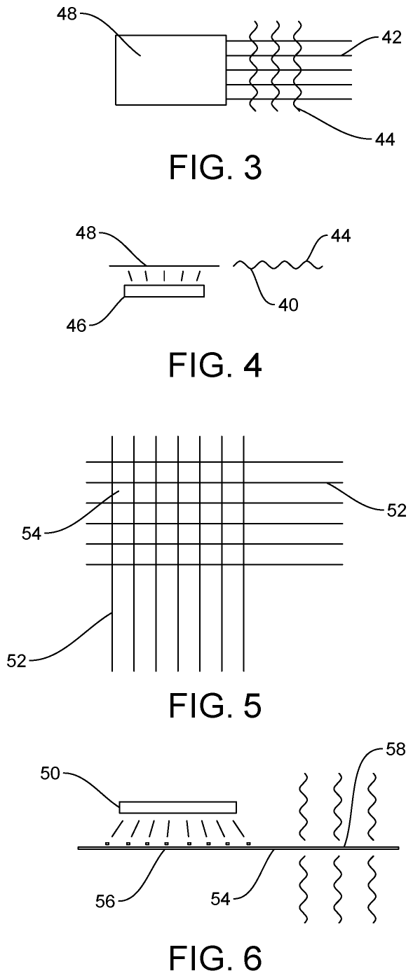

According to another embodiment shown in FIGS. 3 and 4, the ends 40 of multiple fiber optic cables 42 that form part of a fabric 44 are arranged along an edge of a plate-shaped header 48 to receive UV Light emitted into a face of the plate from a light source 46.

According to another embodiment shown in FIGS. 5 and 6, a UV light source or a header 50 emits light into the sides of multiple fiber optic cables 52 at a first location 56 that forms part of a fabric 54. The light received by the sides of the cables 52 is transmitted along a length of the cables 52 to another section of the fabric 54 from which light is emitted from the sides of the cables 52 at a second location 58 remote from the first location 56. Thus, a part of the cables 52 are adapted to receive light via their exposed sides along a length thereof, another part of the cables 52 are adapted to transmit the light without emitting light, and a further part of the cables 52 are adapted to emit the light from a side thereof.

The foregoing description and specific embodiments are merely illustrative of the principles thereof, and various modifications and additions may be made to the fabric by those skilled in the art, without departing from the spirit and scope of this invention.

We claim:

1. A fabric, comprising:
a flexible fiber optic array extending within a body of the fabric; and
a light source affixed to the fabric and arranged to direct electromagnetic radiation into the flexible fiber optic array;
said flexible fiber optic array including at least one fiber optic cable that is configured to transmit the electromagnetic radiation and to emit the electromagnetic radiation from a longitudinally-extending side of the at least one fiber optic cable along a length thereof.

2. The fabric according to claim 1, wherein said longitudinally extending side of said at least one fiber optic cable is remote from ends of said at least one fiber optic cable.

3. The fabric according to claim 2, wherein said at least one fiber optic cable includes a length in which the electromagnetic radiation is transmitted and not emitted, and a length in which the electromagnetic radiation is emitted from said longitudinally extending side.

4. The fabric according to claim 1, wherein said at least one fiber optic cable includes a length in which the electromagnetic radiation is received into said at least one fiber optic cable from said light source via a longitudinally extending side of said at least one fiber optic cable.

5. The fabric according to claim 1, wherein said at least one fiber optic cable includes a bundle of fiber optic cables of which individual fiber optic cables of said bundle are spread out and woven into the fabric and from which the electromagnetic radiation is emitted from longitudinally extending sides thereof from a face of the fabric.

6. The fabric according to claim 1, wherein said at least one fiber optic cable includes multiple fiber optic cables extending in a lattice configuration within the fabric.

7. The fabric according to claim 1, wherein the fabric is comprised of the flexible fiber optic array and at least one other material.

8. The fabric according to claim 1, wherein the at least one fiber optic cable is woven with at least one other material to form the fabric.

9. The fabric according to claim 1, wherein said light source is a light emitting diode.

10. The fabric according to claim 1, wherein said light source is arranged to emit light into an end of said at least one fiber optic cable.

11. The fabric according to claim 1, wherein said light source is arranged to emit light into a longitudinally-extending side of said at least one fiber optic cable remote from an end of said at least one fiber optic cable.

12. The fabric according to claim 1, further comprising a header for transmitting light from the light source to said at least one fiber optic cable.

13. The fabric according to claim 12, wherein said header is a fiber optic cable of greater diameter than that of said at least one fiber optic cable of said flexible fiber optic array.

14. The fabric according to claim 12, wherein said header is plate-shaped having a face and an edge, wherein the light source is configured to emit the electromagnetic radiation into said face of said header and said edge of said header is configured to transmit the electromagnet radiation into said at least one fiber optic cable.

15. The fabric according to claim 1, wherein the electromagnetic radiation is light.

16. The fabric according to claim 15, wherein the light is ultraviolet (UV) light.

17. The fabric according to claim 16, wherein the UV-light is UV-C light for use in sanitizing an object adjacent to, covered by, or surrounded by the fabric.
